(19) United States
(12) Patent Application Publication
(10) Pub. No.: US 2006/0182786 A1
(43) Pub. Date: Aug. 17, 2006

(54) Transmucosal form of administration with reduced mucosal irritation

(75) Inventor: Tina Rademacher, Bad Breisig (DE)

Correspondence Address: D. PETER HOCHBERG CO. L.P.A., 1940 EAST 6TH STREET, CLEVELAND, OH, 44114, US

(73) Assignee: LTS LOHMANN THERAPIE-SYSTEME AG, Andernach (DE)

(21) Appl. No.: 10/562,422

(22) PCT Filed: Jun. 19, 2004
(86) PCT No.: PCT/EP04/06659

§ 371 Date: Dec. 27, 2005

(30) Foreign Application Priority Data
Jun. 27, 2003 (DE) 103 28 942.9

(51) Int. Cl. A61K 9/70

(52) U.S. Cl. 424/443

(57) ABSTRACT

The present invention relates to film-shaped administration forms for transmucosal administration of active substances to the human or animal body. The administration forms are characterized by an adaptation or approximation of the pH value of the base mass intended for the production of the administration form to the physiological pH value of the mucosa to which the administration form is to be applied, said base mass comprising a solvent or a mixture of solvents, at least one matrix-forming polymer and at least one active substance. The invention further comprises a process for the production of such preparations, and the use thereof as administration forms, particularly for pharmaceutical active substances, such that when the resultant administration forms are used, an irritation of the mucous membrane is reduced or even prevented.

### CROSS-REFERENCE TO RELATED APPLICATIONS

This application is a National Stage application of International Application No. PCT/EP2004/006659, filed on Jun. 19, 2004, which claims priority of German application number 103 28 942.9, filed on Jun. 27, 2003.

### BACKGROUND OF THE INVENTION

1. Field of the Invention

The present invention relates to film-shaped preparations which are intended for transmucosal administration of active substances to the human or animal body and upon whose use irritation of the mucosa is reduced or even prevented. The invention further comprises processes for the manufacture of such preparations as well as the use thereof as administration forms, especially for pharmaceutical active substances.

2. Description of the Prior Art

An advantage of transmucosal administration of active substances is that the gastrointestinal route is bypassed, which means that the “first pass” effect after peroral administration, i.e. the metabolism of a significant portion of the active substance during the first liver passage following absorption of the active substance in the gastrointestinal tract, is avoided.

Transmucosal administration forms may be present in the form of pellets, capsules or tablets. Particularly advantageous administration forms for transmucosal administration of active substances are film-like preparations; these are preferably applied in the form of thin lamellae or wafer-shaped objects (“wafers”).

Among other things, the film-shaped administration forms lead to an increase in compliance since their application does not require particularly great discipline. Because of the small layer-thickness of film-shaped administration forms, the persons treated generally do not feel disturbed by the application of the administration form.

The transmucosal administration of active substances may be effected by active substance-containing films which are adhered to the mucosa as mucoadhesive administration forms. In the contact area of the application surface, the active substance can be released to the mucous membrane directly from the administration form. When application takes place in the oral cavity, for example, it is also possible for the active substance contained in the administration form to be released to the surrounding saliva during the period of application, and subsequently to be absorbed by the oral mucosa.

Mucoadhesive administration forms in the form of thin lamellae or wafer-like objects are preferably applied to the oral mucosa, especially sublingually or buccally, to which they adhere on account of their mucoadhesive properties. Furthermore, other mucosal surfaces may also be taken into consideration as an application site, e.g. the nasal mucosa.

During application, the film-like administration form may also, as the case may be, absorb saliva, and the active substance contained in the administration form may get to the outside by diffusion. In this case, it is advantageous that the active substance is released to the saliva after only a very short time lag, so that the saliva-active substance mixture immediately reaches all regions of the oral mucosa and can be absorbed there. The amount of saliva in which the active substance which has been released is dissolved or dispersed per unit of time is relatively small and no excessive flow of saliva results, so that swallowing of the active substance (involving the above-mentioned disadvantages connected with gastrointestinal absorption) can be largely excluded.

Active substance-containing film-shaped administration forms intended for transmucosal administration of active substances may be configured so as to disintegrate in liquids. Upon application of such an administration form, the active substance is present at the mucosa in a very high local concentration. Because of the high thermodynamic pressure which is built up in this way, the active substance rapidly becomes available systemically or locally. Because of their small layer thickness and their disintegratability or dissolvability, these film-shaped, flat administration forms are especially suitable for a very rapid release of medicaments and other active substances, particularly in the oral cavity.

However, distinct irritations of mucous membranes have been observed in those cases where film-shaped administration forms were transmucosally applied in order to administer active substances, particularly where mucoadhesive and disintegratable administration forms were administered. These irritations became manifest in an intense redness of the application site which in some cases lasted for more than 24 hours and in several cases disappeared only after more than 48 hours. It has even been established in histologic studies that cell damage occurred following repeated application of film-shaped administration forms.

For safety considerations, mucous membrane irritations and cell damage after application of film-shaped administration forms are, however, unacceptable and such transmucosal administration forms would not meet regulatory demands.

### SUMMARY OF THE INVENTION

It is therefore an object of the present invention to provide a formulation for film-shaped administration forms intended for transmucosal administration of active substances which avoids irritation of the mucosa, or at least reduces such irritation.

Starting from the following preconsiderations, the above object has been solved by specifically adjusting the pH value in the polymer mass used for the production of film-shaped preparations, i.e., by approximating or adjusting the pH value to the physiological pH value of the mucous membrane to which the administration form is to be applied, so that the pH value of the polymer mass does not, or not significantly, differ, from the physiological pH value of the mucous membrane to which the administration form is to be applied.

Usually, to produce film-shaped preparations, initially a base mass is prepared comprising a solvent or solvent mixture, at least one matrix-forming polymer and at least one active substance, as well as, possibly, further adjuvants which fulfil different functions in the mass or in the dried film, which mass is then extended or extruded to form moist films, by using suitable tools. The moist films are subsequently dried and singularized.

As the solvent, or as one of the solvents of the solvent mixture, water is used with preference.

A pharmaceutical active substance is generally added as a solid phase, in which case frequently a salt of that pharmaceutical active substance is utilized, and less frequently the free base thereof. Hydrochlorides are preferably deployed as active substance salts, but other salts such as citrates or salicylates may also be used. The active substance salts may, furthermore, be present as anhydrates or in hydrated forms.

The cation of active substance salts is often present as a protonated base which in solution dissociates to a lesser or greater extent—depending on the pKa value. Dissociation leads to an increase in the concentration of hydronium ions and thereby to the lowering of the pH. This pH shift to the acid range occurs frequently in the production of materials for film-shaped administration forms.

The conditions present in the moist film are fixed when the spread film has been dried. If this dried film comes into contact with moisture, the conditions that prevailed when the mass was being produced will reappear. As a consequence, it is possible that the pH value at the site of application may be changed as well if the pH of the film clearly deviates from the physiological pH value of the mucous membrane, and this may lead to the mucous membrane irritations observed, especially if the local pH falls distinctly below the physiological pH of the mucous membrane. This is the case if the mass has a pH value during its manufacture which is considerably lower than the physiological pH of the mucous membrane with which the film is brought into contact.

The object of providing film-shaped administration forms which are intended for transmucosal administration of active substances and upon whose application irritation of the mucous membrane is reduced or even prevented, is achieved essentially by approximating or adapting the pH value of the basic mass used for the film-shaped preparation specifically to the physiological pH value of the mucous membrane which comes into consideration for application.

For example, the pH of the oral mucosa in herbivores, such as horses or cattle, is around 8 to 9 and that in humans approximately between 5.5 and 6.5. The pH of the human nasal mucosa is around 8, and the human vaginal mucosa has a pH of around 4.

By adding, e.g., potassium hydroxide, sodium hydroxide or ammonia, the pH value of the base mass for the film-like preparation can be increased, or by adding of, for example, hydrochloric acid or phosphoric acid, lowered. Thus, by titrating alkaline or acidic substances, the pH value of the base mass can be adjusted such that after application of the dry film to a mucosa there occurs no or only a very small change of the local physiological pH, with the result that subsequently no or only a marginal irritation of the skin is observed.

In one particular embodiment, the pH of the polymer mass can also be adjusted to the intended pH with the aid of a physiological buffer system, such as a phosphate buffer.

When adjusting the pH, care has to be taken that no precipitation of the active agent, which is generally present in salt form, occurs. When adjusting the pH, there is a possibility of the active substance base forming, which base does not or only very sparingly redissolve in an aqueous medium, so that at least part of the active substance is bound as a base and is no longer available as an active component in the film-shaped administration form.

### DETAILED DESCRIPTION OF THE INVENTION

In a preferred embodiment, the administration form according to the present invention is mucoadhesive and may have a polymer matrix that serves as an active substance reservoir and has mucoadhesive properties. The administration form may, in the simplest case, consist of a single layer or it may comprise a plurality of layers. In the case of a multilayer structure, at least one of the layers contains an active substance and at least one layer or at least one surface of the administration form possesses mucoadhesive properties.

The polymer matrix of a mucoadhesive administration form preferably contains one or more polymers that are water-soluble and/or capable of swelling in aqueous media. By selecting such polymers it is possible to influence the mucoadhesive properties and the release behaviour.

In another preferred embodiment, the inventive administration form, also including the mucoadhesive embodiment, is configured so as to be disintegratable. These pharmaceutical preparations comprise a matrix which is disintegratable in aqueous media, the matrix being formed of at least one matrix-forming polymer and containing at least one active substance dissolved or dispersed therein. An essential feature of this embodiment consists in that after having been introduced in an aqueous medium or in body fluids it disintegrates rapidly, that is, the disintegration process is substantially completed within 15 minutes, provided that the pharmaceutical form was surrounded during this time by an aqueous medium, e.g. a body fluid. According to preferred embodiments of the invention, the pharmaceutical forms are configured such that they disintegrate within 3 minutes, and with particular preference within 60 seconds, following their introduction into an aqueous medium.

Following application of the pharmaceutical product to the surface of a mucous membrane and its adherence thereto, the pharmaceutical product begins to disintegrate upon action of moisture or of the surrounding aqueous medium, e.g. body fluids; for example, by forming a gel or a solution. Simultaneously, the active substance contained in the pharmaceutical product is released and can now be absorbed directly via the mucous membrane in question, e.g. the oral mucosa.

The mucoadhesive properties and/or the disintegration properties are determined essentially by the type of the matrix-forming polymer/polymers, as well as by the relative portions of these polymers in the preparation.

Suitable as matrix-forming polymers which can be components of a formulation according to the invention are preferably the following water-soluble or at least partially water-soluble polymers—not excluding any other suitable raw materials:

Polyvinyl alcohol (e.g. MOWIOL®); cellulose derivatives such as hydroxypropyl methyl cellulose, hydroxypropyl cellulose, sodium carboxymethyl cellulose (e.g. WALOCEL®), methyl cellulose, hydroxyethyl cellulose and hydroxypropyl ethyl cellulose; starch and starch derivatives; gelatine (various types); polyvinyl pyrrolidones; gum arabic; pullulan; acrylates.

In addition, polymers from the following group are particularly suitable as water-soluble or swellable polymers: dextran; cellulose derivatives, such as carboxymethyl cellulose and ethyl or propyl cellulose; polyacrylic acid, polyacrylates, polyethylene oxide polymers, polyacrylamides, polyethylene glycol, collagen, alginates, pectins, tragacanth, chitosan, alginic acid, arabinogalactan, galactomannan, agar-agar, agarose, carrageenan, and natural gums.

The polymer portion contained in an administration form of the invention preferably amounts to 5 to 95%-wt., especially preferably 15 to 75%-wt., relative to the dry mass of the administration form.

The film-like preparations are advantageously suitable as administration forms for administering pharmaceutical active substances. Therefore, according to a preferred embodiment, such a preparation contains a pharmaceutical active substance or a combination of two or more pharmaceutically active substances. The active agent(s) may be present in dissolved, dispersed, suspended or emulsified form.

Optionally, further releasable substances may be contained, such as aroma substances or sweeteners.

Suitable as active substances are those compounds which are therapeutically effective in humans or animals—without exclusion of any other compounds. Such compounds may come from the following groups: agents for treating infections; virostatics; analgesics such as fentanyl, sufentanil, buprenorphine; anaesthetics; anorectics; active agents for treating arthritis and asthma, such as terbutaline; anticonvulsives; antidepressives; antidiabetics; antihistaminics; antidiarrhoeal agents; agents active against migraine, itching, nausea and retching, travelling sickness or sea-sickness, such as scopolamine and ondansetron; antineoplastic agents; anti-Parkinson agents; antipsychotics; antipyretics; antispasmodics; anticholinergics; agents active against ulcer, such as ranitidine; sympathomimetics; calcium channel blockers, such as nifedipine; beta-blockers; beta-agonists, such as dobutamine and ritodrine; anti-arrhythmic agents; antihypertonics, such as atenolol; ACE inhibitors, such as enalapril; benzodiazepine agonists, such as flumazenil; coronary, peripheral and cerebral vasodilators; stimulants of the central nervous system; hormones; hypnotics; immunosuppressants; muscle relaxants; prostaglandins; proteins; peptides; psychostimulants; sedatives; tranquilizers.

Furthermore, suitable active substances are found in the active substance groups of the parasympatholytics (e.g. scopolamine, atropine, berlactyzine) the parasympathomimetics, the cholinergics (e.g. physostigmine, nicotine), the neuroleptics (e.g. chlorpromazine, haloperidol), the monoamine oxidase inhibitors (e.g. tranylcypromine, selegiline), the sympathomimetics (e.g. ephedrine, D-norpseudoephedrine, salbutamol, fenfluramine), the sympatholytics and antisympathotonic agents (e.g. propranolol, timolol, bupranolol, clonidine, dihydroergotamine, naphazoline), the anxiolytics (e.g. diazepam, triazolam), the local anaesthetics (e.g. lidocaine), the central analgesics (e.g. fentanyl, sufentanil), the antirheumatics (e.g. indomethacin, piroxicam, lornoxicam), the coronary therapeutics (e.g. glycerol trinitrate, isosorbide dinitrate), the estrogens, gestagens and androgens, the antihistaminics (e.g. diphenhydramine, clemastine, terfenadine), the prostaglandin derivatives, the vitamins (e.g. vitamin E, cholecalciferol), the cytostatics, and the cerebroactive glycosides such as digitoxin and digoxin, for example.

The active substance content preferably amounts to 0.1 to 50%-wt, especially preferably 0.5 to 20%-wt., relative to the dry mass of the administration form. A single administration form preferably contains 0.5 to 20 mg, especially preferably 1 to 10 mg, of active substance.

The administration forms according to the invention may optionally contain one or more additives from the following groups: fillers, colourants, flavourings, aroma substances, fragrant substances, emulsifiers, plasticizers, sweeteners, preservatives, permeation-enhancing substances, and antioxidants. Substances suitable for this purpose are in principle known to those skilled in the art.

Addition of flavourings, fragrant substances and aroma substances, either alone or in combination, is particularly advantageous since this increases acceptance of the pharmaceutical preparation in the case of direct oral application. It is, for example, possible to improve the taste impression by adding a refreshing flavouring agent (e.g. menthol, eucalyptol). An unpleasant smell or taste caused by the medicinal active agent can be covered by adding a suitable flavouring or aroma substance. At the same time, this enables a person to take the medicament in an inconspicuous manner since it smells like a refreshing sweet. This additionally contributes to improved compliance.

Particularly suitable are, for instance, flavouring agents and aroma substances from the group comprising menthol, eucalyptol, limonene, phenyl ethanol, camphene, pinene, seasoning aromas such as n-butyl phthalide or cineol, as well as eucalyptus and thyme oil, methyl salicylate, turpentine oil, camomile oil, ethyl vanillin, 6-methyl coumarin, citronellol and acetic acid n-butyl ester.

In the veterinary field, in particular, it is possible to take into account the known preferences of the treated animals when selecting aroma substances. It is, for example, known that cheese, cream and valerian aromas can be advantageously used in pharmaceutical preparations that are intended to be administered to cats. In addition, meat, sausage and fish aromas can be advantageously used in order to increase an animal's readiness to take a medical preparation orally. For certain groups of animals, however, fruit or herb aromas, such as banana, strawberry, mint, cocoa, nut or coffee flavours, are particularly suitable. Mixtures of various flavours may likewise be used.

The film-shaped preparations of the invention may, however, also be used only to release one or more aroma substances, such as menthol or lemon aroma, in the oral cavity, that is, without a pharmaceutical active substance being necessarily contained in the preparation.

The content of aroma substance(s) is preferably 0.1 to 20%-wt., especially preferably 1 to 10%-wt., always relative to the dry mass of the film-shaped administration form.

Substances from the following groups may advantageously be used as further auxiliary substances: filling agents, such as SiO₂; colourants, such as quinoline yellow or TiO₂; disintegrants or wicking agents, which draw water into the matrix and burst the matrix from within, such as aerosil; emulsifiers, such as TWEEN® (polyethoxylated sorbitan fatty acid esters), BRIJ® (polyethoxylated fatty alcohols); sweeteners, such as aspartame, sodium cyclamate and/or saccharine; plasticizers such as PEG (polyethylene glycol) or glycerine; preservatives such as, for example, sorbic acid or its salts.

The proportion of these adjuvants may amount to up to 30%-wt., preferably 1 to 20%-wt., in each case relative to the dry mass of the administration form.

According to a preferred embodiment, the preparations according to the invention contain at least one aroma substance and/or at least one sweetener and/or at least one plasticizer.

The total thickness of the preparations of the invention, particularly of the wafers, is preferably 5 μm to 10 mm, more preferably 50 μm to 2 mm, and especially preferably 0.1 mm to 1 mm. To avoid any foreign body sensation, the layer thickness of the mucoadhesive embodiments should be as small as possible, preferably smaller than 0.2 mm.

The wafers may advantageously be of round, oval, elliptical, triangular, rectangular or polygonal shape, but they may also be of any rounded shape.

The above mentioned wafers are comparatively dense bodies and are preferably of a density between 0.3 g/cm³ and 1.7 g/cm³, especially preferably between 0.5 g/cm³ and 1.5 g/cm³, and most preferably between 0.7 g/cm³ and 1.3 g/cm³.

To achieve specific effects, the administration forms of the invention may be made up of two or more layers. The individual layers may differ from one another in respect of one or more of the following parameters: polymer composition, active substance content, active substance concentration, content of additives.

The surface of the preparations of the invention is typically smooth. It may, however, be advantageous to provide the surface with elevations and depressions, e.g. in form of naps or grooves.

The invention also encompasses preparations of the above-mentioned type which are present in the form of thin, solid foams. Wafers in the form of thin foams are advantageous since they quickly adhere on account of their large specific surface but also disintegrate quickly. The density of these solidified foams is preferably between 0.01 g/cm³ and 0.8 g/cm³, especially preferably between 0.08 g/cm³ and 0.4 g/cm³, and most preferably between 0.1 g/cm³ and 0.3 g/cm³. The calculation of the density is based on the volume filled or enclosed by the entire body of the foam.

In the context of the present invention, the term “aqueous media” is understood to mean, in particular: water, aqueous solutions, suspensions, dispersions, aqueous solvent mixtures as well as physiological liquids and body fluids (e.g. secretory products of the body, saliva, mucus).

### EXAMPLE

An adhesive preparation for transbuccal release of an active substance was tested within the framework of a project in veterinary medicine. The composition of the mucoadhesive preparation, which is indicated in Table 1, was selected such that the preparation disintegrates in an aqueous medium within a few minutes and forms an adhesive gel.

TABLE 1

| Component | Proportion dry mass %-wt. |
|---|---|
| Water/alcohol (1:1) | |
| WALOCEL ® CRT 30 | 38% |
| Active substance hydrochloride | 20% |
| Propanediol | 10% |
| Menthol | 10% |
| Dexpanthenol | 10% |
| Sorbitol | 10% |
| Aroma substance | 2% |

The original pH value of the base mass for this preparation was 5.3. After application of this preparation to the oral mucosa of horses, rather substantial skin irritations occurred with delays in time.

Application of a preparation of the same composition and wherein the pH of the base mass had been raised to 6.1 led to no or only very slight irritations of the mucosa in the treated horses.

Thus, by raising the pH value of the base mass, it was possible to reduce or prevent irritation of the mucosa. The degree of skin irritation correlated with the pH of the polymer mass used to prepare the preparation and thereby with the difference between the pH value of the polymer mass and the physiological pH value of the oral mucosa.

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1. A film-shaped administration form for transmucosal administration of at least one active substance comprising:
a base mass for producing said administration form, said base mass comprising a solvent or a mixture of solvents, at least one matrix-forming polymer and at least one active substance, said base mass having a pH value, wherein
said administration form is a dried film, and wherein
the pH value of the base mass for producing said administration form having been approximated or adapted to the physiological pH value of the mucosa to which the administration form is to be applied during the production of said administration form and wherein
said at least one active substance is selected from the group consisting of pharmaceutically active substances and aroma substances.

2. The administration form according to claim 1, wherein said solvent or at least one solvent of said mixture of solvents is water.

3. The administration form according to claim 1, wherein the matrix-forming polymer is selected from the group consisting of polyvinyl alcohol; cellulose derivatives; starch and starch derivatives; gelatine; polyvinyl pyrrolidones; gum arabic; pullulan; acrylates; dextran; polyacrylic acid; polyacrylates; polyethylene oxide polymers; polyacrylamides; polyethylene glycol; collagen; alginates; pectins; tragacanth; chitosan; alginic acid; arabinogalactan; galactomannan; agar-agar; agarose; carrageenan; and natural gums.

4. The administration form according to claim 1, wherein the polymer portion is 5 to 95%-wt. relative to the dry mass of the administration form.

5. The administration form according to claim 1, wherein the content of said pharmaceutically active substance is 0.1 to 50%-wt., relative to the dry mass of the administration form.

6. The administration form according to claim 1, wherein the content of said aroma substance is 0.1 to 20%-wt., relative to the dry mass of the administration form.

7. The administration form according to claim 1, wherein the pH value of the base mass having been approximated or adapted to a value in the range between 5 and 9.

8. The administration form according to claim 1, wherein the pH value was approximated or adapted by using a chemical selected from the group consisting of sodium hydroxide, potassium hydroxide, ammonia, hydrochloric acid, phosphoric acid and a buffer system.

9. The administration form according to claim 1, wherein said said administration form is mucoadhesive.

10. The administration form according to claim 1, wherein said administration form is disintegratable.

11. The administration form according to claim 10, wherein said administration form disintegrates within 15 minutes after having been introduced in an aqueous medium.

12. Administration form according to claim 1, wherein said administration form administration form is multilayered.

13. The administration form according to claim 1, wherein said administration form contains at least one adjuvant selected from the group consisting of filling agents, colourants, flavourings, aroma substances, fragrant substances, emulsifiers, plasticizers, sweeteners, preservatives, permeation-enhancing substances, and antioxidants.

14. The administration form according to claim 13, wherein the portion of said at least one adjuvant amounts to up to 30%-wt. relative to the dry mass of the administration form.

15. Use of the administration form according to claim 1 for at use selected from the group of applications consisting of oral, gingival, vaginal and rectal application.

16. A process for the production of a film-shaped administration form for transmucosal administration of at least one active substance, comprising the steps of:
preparing a base mass comprising a solvent or a mixture of solvents, at least one matrix-forming polymer and at least one active substance;
approximating or adapting the pH value of the base mass to the physiological pH value of the mucous membrane to which the administration form is to be applied;
extruding the base mass to form a moist film;
drying the moist film; and
singularizing the administration form; wherein
said at least one active substance is selected from the group consisting of pharmaceutically active substances and aroma substances.

17. The process according to claim 16, comprising the step of using water as the solvent or as at least one of the solvents of the mixture of solvents.

18. The process according to claim 16, wherein the step of approximating or adapting the pH value of the base mass comprises the step of adjusting the pH value of the base mass to a value in the range between 5 and 9.

19. The process according to claim 18, wherein the step of adjusting the the pH value is accomplished by adding a chemical selected from the group consisting of sodium hydroxide, potassium hydroxide, ammonia, hydrochloric acid, phosphoric acid and a buffer system.

20. The administration form according to claim 3, wherein said cellulose derivatives are selected from the group consisting of such as hydroxypropyl methyl cellulose, hydroxypropyl cellulose, sodium carboxymethyl cellulose, methyl cellulose, hydroxyethyl cellulose, hydroxypropyl ethyl cellulose, carboxymethyl cellulose, ethyl cellulose and propyl cellulose.

21. The administration form according to claim 4, wherein the polymer portion is 15 to 75%-wt. relative to the dry mass of the administration form.

22. The administration form according to claim 5, wherein the content of said pharmaceutically active substance is 0.5 to 20%-wt. relative to the dry mass of the administration form.

23. The administration form according to claim 6, wherein the content of said aroma substance is 1 to 10%-wt. relative to the dry mass of the administration form.

24. The administration form according to claim 7, wherein the pH value of the base mass was adjusted to a value in the range between 6 and 8.5.

25. The administration form according to claim 24, wherein the pH value of the base mass was adjusted to a value in the range between 6.5 and 8.

26. The administration form according to claim 8, wherein said buffer system is a phosphate buffer.

27. The administration form according to claim 11, wherein said administration form disintegrates within 3 minutes after having been introduced in an aqueous medium.

28. The administration form according to claim 27, wherein said administration form disintegrates within 60 seconds after having been introduced in an aqueous medium.

29. The administration form according to claim 13, wherein the portion of said at least one adjuvant amounts to up to 1 to 20%-wt. relative to the dry mass of the administration form.

30. The process according to claim 18, comprising the step of adjusting the pH value of the base mass to a value in the range between 6 and 8.5.

31. The process according to claim 30, comprising the step of adjusting the pH value of the base mass to a value in the range between 6.5 and 8.

32. The process according to claim 19, wherein said buffer system is a phosphate buffer.
